(19) United States
(12) Patent Application Publication
(10) Pub. No.: US 2026/0240643 A1
(43) Pub. Date: Aug. 20, 2026

(54) ACCOMMODATING INTRAOCULAR LENSES AND METHODS OF MANUFACTURING THE SAME

(71) Applicant: Shifamed Holdings, LLC, Campbell, CA (US)

(72) Inventors: Claudio Argento, Campbell, CA (US); Tom Saul, Portland, OR (US)

(21) Appl. No.: 19/160,415

(22) PCT Filed: Feb. 28, 2024
(86) PCT No.: PCT/US2024/017704

§ 371 Date: Aug. 27, 2025

Related U.S. Application Data
(60) Provisional application No. 63/487,537, filed on Feb. 28, 2023.

(51) Int. Cl. A61F 2/16

(52) U.S. Cl. CPC A61F 2/1624; A61F 2/1648; A61F 2240/001

(57) ABSTRACT

The present technology relates to accommodating intraocular lenses (AIOLs) and methods of manufacturing the same. In many of the embodiments disclosed herein, the AIOLs include an adjustable lens structure and a fixed lens configured to be removably coupled to the accommodating lens structure. The adjustable lens structure can include a base defining an adjustable lens and a lens-receiving area configured to receive the fixed lens. The adjustable lens can have a dynamically adjustable range of optical powers and/ or depths of field. The fixed lens can have a fixed optical power and/or depth of field. The base of the AIOL can be a unitary body formed using, e.g., rotational molding.

### CROSS-REFERENCE TO RELATED APPLICATION(S)

The present application claims priority to U.S. Provisional App. No. 63/487,537, filed Feb. 28, 2023, the entirety of which is hereby incorporated by reference herein.

### TECHNICAL FIELD

The present technology relates to accommodating intraocular lenses (AIOLs) and methods of manufacturing the same.

### BACKGROUND

Cataracts can affect a large percentage of the worldwide adult population with clouding of the native crystalline lens and resulting loss of vision. Patients with cataracts can be treated by native lens removal and surgical implantation of a synthetic intraocular lens (IOL).

Worldwide, there are millions of IOL implantation procedures performed annually. In the U.S., there are 3.5 million cataract procedures performed, while worldwide there are over 20 million annual procedures performed.

Although IOL implantation procedures can be effective at restoring vision, conventional IOLs have several drawbacks. For example, many prior IOLs are not able to change focus as a natural lens would (known as accommodation). Other drawbacks of conventional IOLs include refractive errors that occur after implantation and require glasses for correcting distance vision, or in other cases the IOLs can be effective in providing good far vision, but patients need glasses for intermediate and near vision.

Several multi-focal IOLs have been developed to address these drawbacks, but they too can have drawbacks. For example, although multi-focal IOLs generally perform well for reading and distance vision, in at least some instances such multi-focal IOLs may cause significant glare, halos, reduced contrast sensitivity, and other visual artifacts.

AIOLs have been proposed to provide accommodative optical power in response to the distance at which a patient views an object. However, such AIOLs are generally still in development and have different drawbacks. For example, prior AIOLs can provide insufficient accommodation after implantation or produce suboptimal refractive correction of the eye. The amount of accommodation of the prior AIOLs can also decrease after implantation in at least some instances. The prior AIOLs can also be too large to be inserted through a small incision of the eye and may require the incision to be somewhat larger than would be ideal. Also, at least some of the prior AIOLs can be unstable when placed in the eye, which can lead to incorrect accommodation and other errors.

Improved implantable intraocular lenses that accommodate with the natural mechanisms of controlling focusing of the eye that overcome at least some of the above deficiencies would be desirable. Ideally, such improved AIOLs would provide increased amounts of accommodation when implanted, provide refractive stability, introduce few if any perceptible visual artifacts, and allow the optical power of the eye to change from far vision to near vision in response to the distance of the object viewed by the patient.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIGS. 1A and 1B are perspective views of an AIOL configured in accordance with embodiments of the present technology.

FIG. 1C is a side cross-sectional of the AIOL taken along line 1C-1C of FIG. 1B.

FIG. 2 is a side cross-sectional view of a base of another AIOL configured in accordance with embodiments of the present technology.

FIG. 3 is a side cross-sectional view of a base of another AIOL configured in accordance with embodiments of the present technology.

### DETAILED DESCRIPTION

The present technology is directed to AIOLs and methods for making and using such devices. In many of the embodiments disclosed herein, the AIOLs include an adjustable lens structure and a fixed lens configured to be removably coupled to the accommodating lens structure. The adjustable lens structure can include a base defining an adjustable lens and a lens-receiving area configured to receive the fixed lens. The adjustable lens can have a dynamically adjustable range of optical powers and/or depths of field. The fixed lens can have a fixed optical power and/or depth of field. The base of the AIOL can be a unitary body formed using, e.g., rotational molding (“rotomolding”). Forming the base as a unitary body is expected to prevent, or at least partially prevent, (i) manufacturing problems/difficulties associated with using adhesive and/or other techniques to join multiple components together to form the base, and/or (ii) variations in material properties associated with different portions of a batch of material or different batches of material used to create the multiple components.

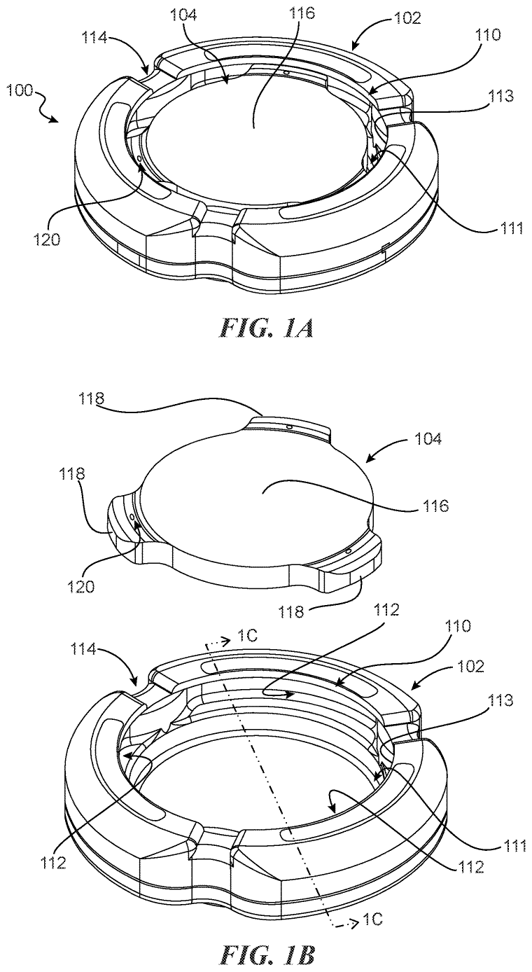

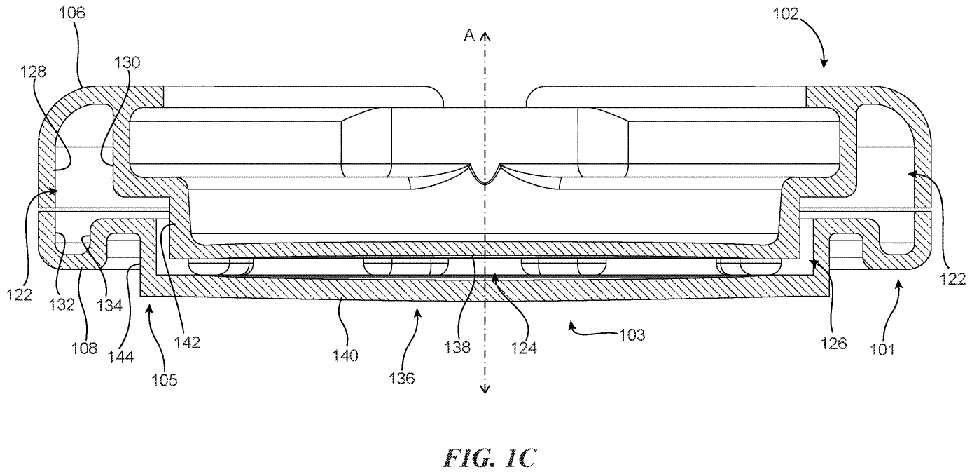

Specific details of various embodiments of the present technology are described below with reference to FIGS. 1A-3. Although many of the embodiments are described below with respect to AIOLs and associated methods, other embodiments are within the scope of the present technology. Additionally, other embodiments of the present technology can have different configurations, components, and/or procedures than those described herein. For instance, AIOLs configured in accordance with the present technology may include additional elements and features beyond those described herein, or other embodiments may not include several of the elements and features shown and described herein.

As used herein, the use of relative terminology, such as “about,” “approximately,” “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

FIGS. 1A and 1B are perspective views of an adjustable intraocular lens 100 (“AIOL 100”) configured in accordance with embodiments of the present technology. The AIOL 100 can include an adjustable or base lens structure 102 (“base 102”) and a fixed power and/or fixed depth of field lens 104 (“fixed lens 104”). The base 102 can include a first or anterior component 106 and a second or posterior component 108, which can be coupled to one another to form the base 102. As described in detail below with reference to FIG. 1C, the first component 106 and the second component 108 can together at least partially define an adjustable lens or optical component (e.g., a lens having an adjustable optical power and/or depth of field) configured to provide an adjustable optical power/correction. The first component 106 of the base 102 can define a concavity or a lens-receiving area or volume 110 (“volume 110”) configured to receive at least a portion of the fixed lens 104. The volume 110 can include a chamber or cavity 111 having an opening 113. The volume 110, the cavity 111, and/or the opening 113 can have one or more dimensions (e.g., diameter, circumference, depth, volume, etc.) corresponding to one or more dimensions of the fixed lens 104, such that the fixed lens 104 can be positioned within the volume 110 by inserting the fixed lens 104 through the opening 113 and into the cavity 111. In some embodiments, the volume 110 can include one or more features configured to secure the fixed lens 104 within the volume 110 and/or otherwise prevent, or at least partially prevent, the fixed lens 104 from inadvertently leaving the volume 110 once the fixed lens 104 is positioned therein. In the illustrated embodiment, for example, the volume 110 includes one or more groove or slots 112 (FIG. 1B) extending radially outward toward an outer perimeter of the base 102, and each of the slots 112 is configured to receive a respective portion of the fixed lens 104.

In some embodiments, the base 102 can include flow-through features 114 that enhance the rate and ease with which Ophthalmic Viscosurgical Devices (OVDs) used during the implantation of AIOLs can be removed from the natural lens capsule. The embodiment of the AIOL 100 illustrated in FIGS. 1A and 1B comprises three outer flow-through features 114. Each of the outer flow-through features 114 can be detents, such as recesses, distributed circumferentially along the perimeter of the base 102. The flow-through features 114 can create passages between the outer perimeter of the AIOL 100 and an inner surface of an eye capsule (not shown) in which the AIOL 100 is implanted to, e.g., allow fluid flow around an outer perimeter of the AIOL 100. In the illustrated embodiment, for example, the flow-through features 114 are formed in peripheral/outer regions of the first component 106 and second component 108. In other embodiments, the flow-through features 114 can have other suitable positions in the first component 106 and/or the second component 108. Although three outer flow-through features 114 are illustrated in FIGS. 1A and 1B, other embodiments may comprise fewer or more of the flow-through features 114 than illustrated. In these and other embodiments, the outer flow-through features 114 may additionally provide rotational constraint to maintain the rotational orientation of the base 102 with respect to a patient's eye capsule when implanted.

The fixed lens 104 can include an optical or lens portion 116 and one or more tabs 118 (FIG. 1B) extending radially outward from the lens portion 116. The lens portion 116 can have a fixed (e.g., positive, negative, or zero) optical power and/or a fixed depth of field. In at least some embodiments, for example, the lens portion 116 includes one or more of an asymmetrically powered lens (e.g., a toric lens), a spherical lens, an aspheric lens, a plano-convex lens, a convex-concave lens, a convex-convex lens, and/or another suitable lens. In some embodiments, the lens portion 116 can have a diopter of between about 0 D and about 20 D, such as between about 8 D and about 20 D, between about 12.5 D and about 18.5 D, any diopter therebetween, or another suitable diopter. In other embodiments, the lens portion 116 can have a negative diopter, e.g., a diopter of between about −5 D and about −0.01 D, any diopter therebetween, or another suitable diopter

At least a portion of one or more of the tabs 118 can be configured to be received by the base 102, for example, to couple or otherwise at least partially or fully prevent movement of the fixed lens 104 relative to the base 102. In the illustrated embodiment, for example, at least a portion of each of the tabs 118 are positioned within a corresponding one of the slots 112 when the fixed lens 104 is positioned within the volume 110 (FIG. 1B). The interaction between the tabs 118 and the slots 112 can prevent, or at least partially prevent, unintended rotation of the fixed lens 104. In at least some embodiments, for example, the fixed lens 104 is expected to maintain a generally or substantially consistent rotational alignment relative to the base 102 unless or until one or both of the fixed lens 104 and the base 102 are manipulated by a practitioner or other user.

In some embodiments, individual ones of the tabs 118 can include one or more apertures or holes 120. Each of the apertures 120 can extend at least partially or fully through the corresponding tab 118, and can be engaged by a surgical tool to manipulate the fixed lens 104, such as during implantation and/or removal of the fixed lens 104. In some embodiments, the apertures 120 are arranged in pairs. In some embodiments, the apertures 120 are distributed in a circumferential pattern. In some embodiments, one or more of the apertures 120 have a different size (e.g., width or diameter) and/or shape than other apertures 120. For example, each of the apertures 120 may have a different size than each of the other apertures 120. Using apertures of varying size can allow for further visual confirmation of the rotational alignment of the fixed lens 104 (e.g., about the optical axis of the AIOL 100). Confirming the alignment/orientation of the fixed lens 104 is expected to reduce the risk that a toric lens or other non-annularly symmetric fixed lens is improperly oriented with respect to the base 102 and/or native eye capsule into which the AIOL 100 is implanted. In some embodiments, the fixed lens 104 can include additional visual markers to indicate an orientation of the fixed lens 104 with respect to the base 102 and/or the native eye capsule.

FIG. 1C is a cross-section of the AIOL 100 taken along line 1C-1C of FIG. 1B. As best seen in FIG. 1C, the base 102 can include bellows or haptic portion 101, a lens or visual correction portion 103, and/or intermediate or transition portion 105. Relative to an optical axis A of the base 102, the bellows portions 101 can be positioned radially outwardly from the lens portion 103. In at least some embodiments, for example, the bellows portion 101 extends continuously circumferentially around the lens portion 103. The transition portion 105 can be positioned at least partially between (e.g., radially between, relative to the optical axis A) the bellows portion 101 and the lens portion 103, e.g., to connect the bellows portion 101 to the lens portion 103. The bellows portion 101 can define a first fluid chamber or reservoir 122 (which can also be referred to as an “outer fluid reservoir 122,” an “outer fluid volume 122,” a “haptic reservoir 122,” and/or the like). The outer fluid reservoir 122 can be defined at least partially between a first or outer wall 128 and a second or inner wall 130 of the first component 106 and/or a first or outer wall 132 and a second or inner wall 134 of the second component 108. The lens portion 103 can define a second fluid chamber or reservoir 124 (which can also be referred to as an “inner fluid reservoir 124,” an “inner fluid volume 124” an “optical fluid chamber 124,” and/or the like). One or both of the bellows and optical portions 101, 103 can be filled with a fluid. The fluid contained can have a refractive index that is less than, equal to, or greater than the refractive index of aqueous and/or another portion of a patient's eye. The transition portion 105 can define a channel 126 that extends between and fluidly couples the outer fluid reservoir 122 and the inner fluid reservoir 124 to allow the fluid to flow between the outer fluid reservoir 122 and the inner fluid reservoir 124. The channel 126 can be defined at least partially between a third or innermost wall 142 of the first component 106 and/or a third or innermost wall 144 of the second component 108.

The outer fluid reservoir 122 can be configured to operate as a bellows to cause fluid flow (e.g., fluid displacement) between the outer fluid reservoir 122 and the inner fluid reservoir in response to forces applied to the AIOL 100. For example, a compressive force applied to a portion of the AIOL 100 can displace fluid within the base 102 by, e.g., causing fluid to flow/redistribute (via the channel 126) between the outer fluid reservoir 122 and the inner fluid reservoir 124. In at least some embodiments, for example, a radially compressive force applied to at least a portion of the AIOL 100, such as an outer perimeter of the base 102, can decrease a volume of the outer fluid reservoir 122 and/or increase a pressure within the outer fluid reservoir 122 and cause fluid within the outer fluid reservoir 122 to flow toward and/or into the inner fluid reservoir 124, which can thereby cause expansion of the inner fluid reservoir. A decrease in the radially compressive force can allow the outer perimeter of the base 102 to expand and thereby increase the volume of the outer fluid reservoir 122 and/or decrease the pressure within the outer fluid reservoir 122. In response, fluid within the inner fluid reservoir 124 can flow toward and/or into the outer fluid reservoir 122, which can thereby return the inner toward reservoir 124 toward and/or to an unexpanded state.

The base 102 can include an adjustable lens 136 at least partially defined by the inner fluid reservoir 124, a first optical portion 138 of the first component 106 of the base 102, and a second optical portion 140 of the second component 108 of the base 102. The adjustable lens 136 can be positioned posterior to the fixed lens 104 and/or the lens portion 116 thereof. One or both of the first optical portion 138 and the second optical portion 140 can be planar members or optical membranes of the first component 106 and the second component 108, respectively. In at least some embodiments, for example, one or both of the first optical portion 138 and the second optical portion 140 can be optical membranes integrally formed with the other portions of the respective first component 106 and the second component 108. In other embodiments, one or both of the first optical portion 138 and the second optical portion 140 can include one or more deformable lenses configured to provide a fixed power that can be positive or negative, and that are configured to deflect in response to changes to a volume and/or fluid pressure within the inner fluid reservoir 124. In at least some embodiments, for example, fluid flow between the outer fluid reservoir 122 and the inner fluid reservoir 124 can cause one or both of the first component 106 and the second component 108 to bend or deflect (in, e.g., a direction parallel, or at least generally parallel to an optical axis of the adjustable lens 136) in response to an increase or decrease in a volume of fluid and/or a fluid pressure within the inner fluid reservoir 124. This increase or decrease in the volume of fluid within the inner fluid reservoir 124 can, in turn, adjust (e.g., increase or decrease) the optical power and/or depth of field of the adjustable lens 136 and/or the range of adjustability of the optical power and/or the depth of field of the adjustable lens 136. In at least some embodiments, for example, the adjustable lens 136 has a diopter of between about 5 D and about 7.5 D, such as between about 5.8 D and about 7.3 D, any diopter therebetween, or another suitable diopter. Additionally, or alternatively, the diopter of the adjustable lens 136 can have a range of adjustment of up to 1.5 D, 3 D, 4 D, 5 D, 7.5 D, any range therebetween, or another suitable range.

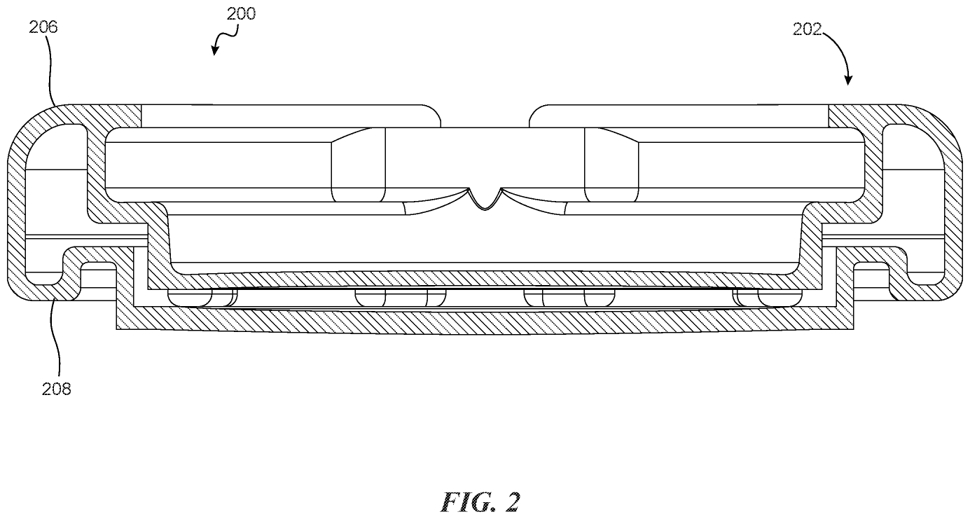

FIG. 2 is a side cross-sectional view of a base 202 of another AIOL 200 configured in accordance with embodiments of the present technology. At least some aspects of the base 202 can be at least generally similar or identical in structure and/or function to the base 102 of FIGS. 1A-1C. However, instead of including first and second components 106/108 (FIG. 1C), the base 202 is a single-piece component, a continuous region, and/or a unitary body including a first (e.g., upper or anterior) portion 206 and a second (e.g., lower or posterior) portion 208. The first portion 206 can be at least generally similar or identical in structure and/or function to the first component 106, and the second portion 208 can be at least generally similar or identical in structure and/or function to the second component 108, but the base 202 can omit the adhesive(s) and/or other bonds used to couple the first component 106 to the second component 108. Compared to embodiments in which AIOL bases are formed by coupling multiple components together with adhesive or other coupling techniques (e.g., thermal welding, solvent welding, plasma bonding, polymerization, etc.), forming the base 202 as a single-piece component is expected to prevent, or at least partially prevent, pre-stressing and/or misalignment that can occur during assembly of the AIOL bases.

The base 202 can be formed via rotomolding by, for example, at least partially filling a mold with one or more materials that polymerize over time and/or during the rotomolding process. In at least some embodiments, for example, the materials can include one or more thermoset and/or thermoplastic materials (e.g., liquid silicone resin, thermoset silicone, urethane, etc.), one or more catalyst-and/or radical-driven polymerizing materials (e.g., silicone, polymethylmethacrylate (“PMMA”), one or more ultraviolet or other light-cured polymers, etc.), and/or other suitable materials. Once the mold is at least partially filled, the mold is placed in a furnace and rotated or spun about one or more axes at a speed and for a predetermined amount of time to, e.g., cause the material to spread out along and/or cover an inner surface of the mold. The speed and/or the predetermined amount of time can be customized to allow the material within the mold to stabilize and/or otherwise conform to the inner surface of the mold. The speed can include, for example, a speed of up to 5 rotations per minute (rpms), 10 rpms, 20 rpms, 50 rpms, 100 rpms, 500 rpms, 1000 rpms, or another suitable speed. The predetermined amount of time can include, for example, a time of up to 1 minute, 5 minutes, 10 minutes, 30 minutes, 1 hour, 6 hours, 12 hours, 24 hours, 48 hours, or another suitable amount of time. In at least some embodiments, the speed can vary (e.g., increase and/or decrease) during the predetermined amount of time. Before, during, and/or after the mold is rotated or spun, heat and/or other energy (e.g., ultraviolet light radiation) can be applied to the mold and/or the material(s) contained therein. In at least some embodiments, for example, the mold can be at least partially transparent and/or otherwise transparent to ultraviolet light, and light from an ultraviolet light source can be applied to the material within the mold through the surface of the mold. In these and/or other embodiments, heat can be applied to the mold and transmitted (e.g., radiantly, and/or via conduction and/or convection) to the material within the mold, and/or one or more heating elements can be positioned within the mold to directly heat the material contained therein.

After the predetermined amount of time has passed, the mold can be removed from the furnace and/or otherwise be allowed to cool and the base 202 can be removed from the mold. An interior portion of the base 202 can be filled with an optical fluid before implantation in an eye of the patient. Additionally, or alternatively, other rotomolding techniques know to those of ordinary skill in the art can be used to form the base 202. Compared to embodiments in which multiple separate components are manufactured separately and later joined to form an AIOL base, forming the base 202 as a single-piece component (via, e.g., rotomolding) in accordance with embodiments of the present technology is expected to increase the consistency/uniformity of the material properties of the base 202 by, e.g., preventing, or at least partially preventing, variations in material properties associated with different portions of a batch of material and/or different batches of material used to create the multiple separate components. The increased consistency/uniformity of the material properties of the base 202 can, in turn, improve the adjustability and/or accommodative response of the adjustable lens of the base 202. In other embodiments, the base 202 can be formed using additive manufacturing and/or any other suitable process or technique, but may still possess one or more of the advantages described above.

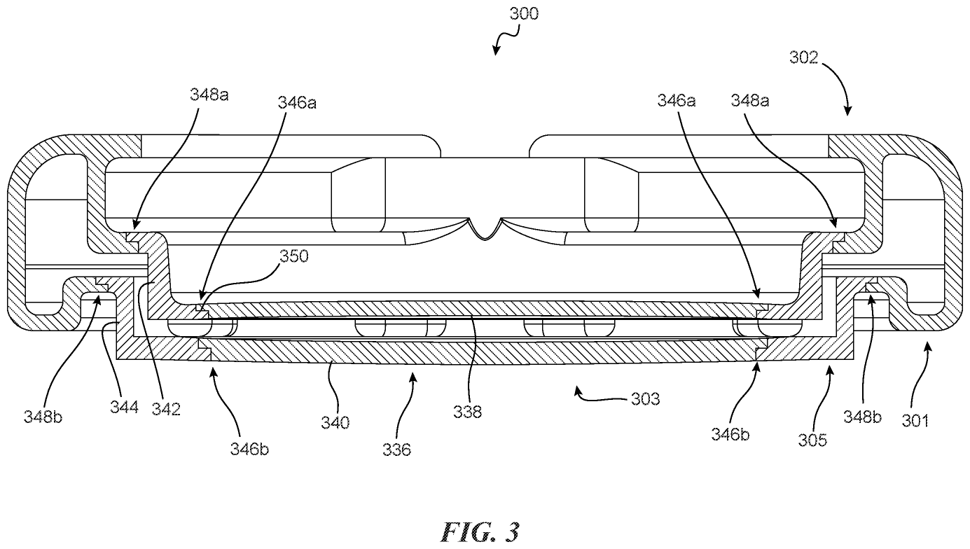

FIG. 3 is a side cross-sectional view of a base 302 of another AIOL 300 configured in accordance with embodiments of the present technology. At least some aspects of the base 302 can be at least generally similar or identical in structure and/or function to the base 102 of FIGS. 1A-1C. For example, the base 302 includes a bellows portion 301, a lens portion 303, and a transition portion 305. One or more of these portions 301, 303, 305 can be manufactured individually and then coupled to one or more of the other portions to form the base 302. In at least some embodiments, for example, the bellows portion 301 can be molded or rotomolded and then the lens portion 303 (including, e.g., first and second optical portions 338, 340) can be bonded or coupled (e.g., via adhesives, mating engagement, mechanical connectors, etc.) to the bellows portion 301. The transition portion 305 (including, e.g., walls 342, 344) can be molded or rotomolded with (e.g., as part of) the bellows portion 301, or can be manufactured separately and bonded or coupled (e.g., via adhesives, mating engagement, mechanical connectors, etc.) to the bellows portion 301 and the optical portion 303. In some embodiments, after molding or rotomolding the bellows portion 301, a separate mold or rotomolding process can be used to mold or rotomold the transition portion 305 and/or the optical portion 303 to the previously molded (or rotomolded) bellows portion 301.

In at least some embodiments, the base 302 includes one or more first joints or seams 346 between the optical portion 303 and the transition portion 305 (individually identified as a primary or anterior first seam 346a between the first optical portion 338 and the wall 342 and a secondary or posterior first seam 346b between the second optical portion 340 and the wall 344) and/or one or more second joints or seams 348 between the transition portion 305 and the bellows portion 301 (individually identified as a primary or anterior second seam 348a between the wall 342 and the bellows portion 301 and a secondary or posterior second seam 348b between the wall 344 and the bellows portion 301). In some embodiments, the second seams 348 can be omitted and the transition portion 3054 can be rotomolded or otherwise formed with the bellows portion 301. In some embodiments, one or more of the seams 346, 348 can have a stepped or shiplap configuration defining an overlap 350 (only labeled for the primary first seam 346a for the purpose of illustrative clarity) between the portions coupled at that seam. The stepped or shiplap configuration of one or more of the seams 346, 348 can increase the surface area of the seams 346, 348 which can increase the strength of the bonding/coupling (e.g., adhesive coupling, plasma bonding, etc.) between the various portions 301, 303, 305 at the respective seams 346, 348.

The AIOL devices described herein may be implanted by preparing the eye and removing the native lens from the capsule in any appropriate manner. The fluid-filled structure may then be placed in the capsule of the eye. The patient may then be evaluated for a base optical power and/or astigmatic correction, and a fixed lens can be selected to provide the desired based power or astigmatic correction for the fluid-filled structure in the implanted state in the capsule of the eye. The specific fixed lens to provide the post-implant base power or astigmatic correction is then inserted into the previously implanted fluid-filled structure of the AIOL. The chosen fixed lens may then be coupled to the fluid-filled structure within the eye capsule. This is possible in the AIOLs of the present technology because the fixed lenses are anteriorly-positioned when implanted, e.g., positioned anterior to the adjustable lens and/or attached to the anterior first component of the AIOLs. As described above, one or more of the fluid-filled accommodating structure or fixed lens may each be flexible such that they may be reconfigured (e.g., folded) to a reduced-profile delivery configuration for delivery into the lens capsule. In some instances, it may be required to make a further correction to the fixed portion after the time of the surgery. Such instance may occur anywhere from days to years after the surgery. At such times, the patient may return to the physician and the fixed lens may be replaced with a new fixed lens having a different optical power or other prescription. In such instances, the new prescription may be characterized prior to or after removal of the original fixed lens. In some instances, the new fixed lens may be fabricated and implanted at the time of the examination, in others the patient may return for implantation of the fixed lens sometime after the examination.

Several embodiments of the present technology are directed to a kit having an accommodating structure and a first fixed lens that has no optical base power. The kit can further include one or more second fixed lenses having various based powers or other optical properties. In practice, the accommodating structure can be implanted into the native eye capsule, and then the first fixed lens can be coupled to the accommodating structure. The optical properties of the implanted accommodating structure can then be assessed in situ with the first fixed lens in place to determine the desired optical properties of the fixed lens. If the optical properties of the assembled accommodating structure and first fixed lens without a base power are appropriate, then the system can remain implanted without additional changes. However, if a different base power or some other optical property is desired (e.g., toric or other asymmetrical optics), then the first fixed lens without a base power can be replaced with a second fixed lens having the desired optical properties based on the optical properties of the implanted accommodating portion with a fixed lens attached.

In some embodiments, the fixed portion of the AIOL may be fabricated from materials different from the accommodating portion. Such materials include hydrophilic or hydrophobic methacrylate or silicones and any other materials traditionally used in non-accommodating IOLs. The fixed lens may be fabricated from materials harder than those used for the accommodating portion. One or both of the accommodating portion/lens and the fixed portion/lens may be machined, cast molded (e.g., reactive cast molded), injected molded, and/or formed by other processes or combinations of processes. Any or all of the structures described herein may be constructed from a transparent or translucent material. For example, the above-described accommodating structures and fixed lenses can be constructed from transparent materials, even if they are illustrated as opaque in the associated figures.

Any of the features of the intraocular lens systems described herein may be combined with any of the features of the other intraocular lenses described herein and vice versa. Additionally, several specific examples of embodiments in accordance with the present technology are set forth below in the following examples.

### EXAMPLES

Several aspects of the present technology are set forth in the following examples:
1. An accommodating intraocular lens assembly, comprising:
a base including an adjustable power lens having an optical power and an optical axis, wherein the base is a single-piece component composed of a polymer,
wherein the optical power of the adjustable power lens changes dynamically along an optical axis of the adjustable power lens in reaction to a radial force on the base in a direction non-parallel to the optical axis; and
a fixed power lens configured to be removably coupled to the base.
2. The accommodating intraocular lens assembly of example 1 wherein the single-piece component base is fabricated via a rotomolding process.
3. The accommodating intraocular lens assembly of example 1 or 2 wherein, when the fixed power lens is coupled to the base, the fixed power lens is aligned with the adjustable power lens along the optical axis.
4. The accommodating intraocular lens assembly of any of examples 1-3 wherein a thickness of the adjustable power lens, in a direction parallel to the optical axis, changes in reaction to radial force on the base in a direction non-parallel to the optical axis.
5. The accommodating intraocular lens assembly of any of examples 1-4 wherein the polymer includes a thermoset and/or thermoplastic material.
6. The accommodating intraocular lens assembly of any of examples 1-5 wherein the polymer includes a ultraviolet light-cured polymer.
7. The accommodating intraocular lens assembly of any of examples 1-6 wherein the base does not include adhesive.
8. The accommodating intraocular lens assembly of any of examples 1-7 wherein the fixed power lens is positioned anterior to the adjustable power lens when the fixed power lens is coupled to the base.
9. A method for manufacturing an adjustable intraocular lens, the method comprising:
at least partially filling a mold configured to form a base of the adjustable intraocular lens with a polymer;
rotating the mold about one or more axes for a predetermined amount of time; and
after the predetermined amount of time has passed, removing the base from mold.
10. The method of example 9 wherein at least partially filling the mold with the polymer includes at least partially filling the mold with a thermoset and/or thermoplastic material.
11. The method of example 9 or 10, further comprising applying ultraviolet light to the polymer within the mold.
12. The method of any of examples 9-11, further comprising applying heat to the mold and, before removing the base from the mold, allowing the mold to cool.
13. The method of any of examples 9-12 wherein rotating the mold includes rotating the mold at a speed of up to 5 rpms, 10 rpms, 20 rpms, 50 rpms, 100 rpms, 500 rpms, or 1000 rpms.
14. The method of any of examples 9-13 wherein rotating the mold for the predetermined amount of time includes rotating the mold for up to 1 minute, 5 minutes, 10 minutes, 30 minutes, 1 hour, 6 hours, 12 hours, 24 hours, or 48 hours.
15. The method of any of examples 9-14 wherein rotating the mold includes causing the polymer to coat at least a portion of an inner surface of the mold.
16. The method of any of examples 9-15, further comprising coupling a fixed power lens to the base, wherein coupling the fixed power lens includes aligning the fixed power lens with an optical axis of an adjustable lens of the base.
17. An accommodating intraocular lens assembly, comprising:
a base including a bellows region and a lens region, wherein—
   the bellows region defines an outer fluid reservoir and is a single-piece component composed of a polymer,
   the lens region includes a first optical portion coupled to the bellows region at a first seam and a second optical portion coupled to the bellows region at a second seam to define an inner fluid volume, and
   the first and second optical portions and the inner fluid volume together define an adjustable power lens having an optical power, wherein the base is configured to dynamically change the optical power in reaction to a force on the base; and
a fixed power lens configured to be removably coupled to the base.
18. The accommodating intraocular lens assembly of example 17, further comprising a transition region positioned between and coupling the bellows region to the lens region, wherein the transition region includes a first wall extending between the first optical portion and the bellows region and a second wall extending between the second optical portion and the bellows region.
19. The accommodating intraocular lens assembly of example 18 wherein the single-piece component includes the bellows region and the transition region.
20. The accommodating intraocular lens assembly of any of examples 17-19 wherein the first seam has a first stepped configuration defining a first overlap between the first optical portion and the bellows region and/or wherein the second seam has a second stepped configuration defining a second overlap between the second optical portion and the bellows region.
21. The accommodating intraocular lens assembly of any of examples 17-20 wherein the single-piece component bellows region is fabricated via a rotomolding process.
22. The accommodating intraocular lens assembly of any of examples 17-21 wherein the fixed power lens is positioned anterior to the adjustable power lens when the fixed power lens is coupled to the base.
23. The accommodating intraocular lens assembly of any of examples 17-22 wherein the polymer includes a thermoset and/or thermoplastic material.
24. The accommodating intraocular lens assembly of any of examples 17-23 wherein the polymer includes an ultraviolet light-cured polymer.
25. A method for manufacturing an adjustable intraocular lens, the method comprising:
at least partially filling a mold configured to form at least a bellows region of a base of the adjustable intraocular lens with a polymer;
rotating the mold about one or more axes for a predetermined amount of time;
after the predetermined amount of time has passed, removing the bellows region from mold; and
coupling a lens portion of the base to the bellows region.
26. The method of example 25 wherein coupling the lens portion of the base to the bellows region includes—
coupling a first optical portion to the bellows region at a first seam; and
coupling a second optical portion to the bellows region at a second seam posterior to the first seam.
27. The method of example 25 or 26 wherein the mold is configured to form the bellows region and a transition region of the base, and wherein coupling the lens portion to the bellows region includes coupling the lens portion to the bellows region via the transition region.
28. The method of any of examples 25-27 wherein at least partially filling the mold with the polymer includes at least partially filling the mold with a thermoset and/or thermoplastic material.
29. The method of any of examples 25-28, further comprising coupling a fixed power lens to the base, wherein coupling the fixed power lens includes aligning the fixed power lens with an optical axis of an adjustable lens of the base.
30. The method of any of examples 25-29, further comprising applying ultraviolet light to the polymer contained within the mold.

31. The method of any of examples 25-30, further comprising applying heat to the mold and, before removing the bellows region from the mold, allowing the mold to cool.

### Conclusion

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the AIOLs described herein may be combined with any of the features of the other AIOLs described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with AIOLs have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An accommodating intraocular lens assembly, comprising:
a base including an adjustable power lens having an optical power and an optical axis, wherein the base is a single-piece component composed of a polymer,
wherein the optical power of the adjustable power lens changes dynamically along an optical axis of the adjustable power lens in reaction to a radial force on the base in a direction non-parallel to the optical axis; and
a fixed power lens configured to be removably coupled to the base.

2. The accommodating intraocular lens assembly of claim 1 wherein the single-piece component base is fabricated via a rotomolding process.

3. The accommodating intraocular lens assembly of claim 1 wherein, when the fixed power lens is coupled to the base, the fixed power lens is aligned with the adjustable power lens along the optical axis.

4. The accommodating intraocular lens assembly of claim 1 wherein a thickness of the adjustable power lens, in a direction parallel to the optical axis, changes in reaction to radial force on the base in a direction non-parallel to the optical axis.

5. The accommodating intraocular lens assembly of claim 1 wherein the polymer includes a thermoset and/or thermoplastic material.

6. The accommodating intraocular lens assembly of claim 1 wherein the polymer includes a ultraviolet light-cured polymer.

7. The accommodating intraocular lens assembly of claim 1 wherein the base does not include adhesive.

8. The accommodating intraocular lens assembly of claim 1 wherein the fixed power lens is positioned anterior to the adjustable power lens when the fixed power lens is coupled to the base.

9. A method for manufacturing an adjustable intraocular lens, the method comprising:
at least partially filling a mold configured to form a base of the adjustable intraocular lens with a polymer;
rotating the mold about one or more axes for a predetermined amount of time; and
after the predetermined amount of time has passed, removing the base from mold.

10. The method of claim 9 wherein at least partially filling the mold with the polymer includes at least partially filling the mold with a thermoset and/or thermoplastic material.

11. The method of claim 9, further comprising applying ultraviolet light to the polymer within the mold.

12. The method of claim 9, further comprising applying heat to the mold and, before removing the base from the mold, allowing the mold to cool.

13. The method of claim 9 wherein rotating the mold includes rotating the mold at a speed of up to 5 rpms, 10 rpms, 20 rpms, 50 rpms, 100 rpms, 500 rpms, or 1000 rpms.

14. The method of claim 9 wherein rotating the mold for the predetermined amount of time includes rotating the mold for up to 1 minute, 5 minutes, 10 minutes, 30 minutes, 1 hour, 6 hours, 12 hours, 24 hours, or 48 hours.

15. The method of claim 9 wherein rotating the mold includes causing the polymer to coat at least a portion of an inner surface of the mold.

16. The method of claim 9, further comprising coupling a fixed power lens to the base, wherein coupling the fixed power lens includes aligning the fixed power lens with an optical axis of an adjustable lens of the base.

17. An accommodating intraocular lens assembly, comprising:
a base including a bellows region and a lens region, wherein—
the bellows region defines an outer fluid reservoir and is a single-piece component composed of a polymer,
the lens region includes a first optical portion coupled to the bellows region at a first seam and a second optical portion coupled to the bellows region at a second seam to define an inner fluid volume, and
the first and second optical portions and the inner fluid volume together define an adjustable power lens having an optical power, wherein the base is configured to dynamically change the optical power in reaction to a force on the base; and
a fixed power lens configured to be removably coupled to the base.

18. The accommodating intraocular lens assembly of claim 17, further comprising a transition region positioned between and coupling the bellows region to the lens region, wherein the transition region includes a first wall extending between the first optical portion and the bellows region and a second wall extending between the second optical portion and the bellows region.

19. The accommodating intraocular lens assembly of claim 18 wherein the single-piece component includes the bellows region and the transition region.

20. The accommodating intraocular lens assembly of claim 17 wherein the first seam has a first stepped configuration defining a first overlap between the first optical portion and the bellows region and/or wherein the second seam has a second stepped configuration defining a second overlap between the second optical portion and the bellows region.

21. The accommodating intraocular lens assembly of claim 17 wherein the single-piece component bellows region is fabricated via a rotomolding process.

22. The accommodating intraocular lens assembly of claim 17 wherein the fixed power lens is positioned anterior to the adjustable power lens when the fixed power lens is coupled to the base.

23. The accommodating intraocular lens assembly of claim 17 wherein the polymer includes a thermoset and/or thermoplastic material.

24. The accommodating intraocular lens assembly of claim 17 wherein the polymer includes an ultraviolet light-cured polymer.

25. A method for manufacturing an adjustable intraocular lens, the method comprising:
at least partially filling a mold configured to form at least a bellows region of a base of the adjustable intraocular lens with a polymer;
rotating the mold about one or more axes for a predetermined amount of time;
after the predetermined amount of time has passed, removing the bellows region from mold; and
coupling a lens portion of the base to the bellows region.

26. The method of claim 25 wherein coupling the lens portion of the base to the bellows region includes—
coupling a first optical portion to the bellows region at a first seam; and
coupling a second optical portion to the bellows region at a second seam posterior to the first seam.

27. The method of claim 25 wherein the mold is configured to form the bellows region and a transition region of the base, and wherein coupling the lens portion to the bellows region includes coupling the lens portion to the bellows region via the transition region.

28. The method of claim 25 wherein at least partially filling the mold with the polymer includes at least partially filling the mold with a thermoset and/or thermoplastic material.

29. The method of claim 25, further comprising coupling a fixed power lens to the base, wherein coupling the fixed power lens includes aligning the fixed power lens with an optical axis of an adjustable lens of the base.

30. The method of claim 25, further comprising applying ultraviolet light to the polymer contained within the mold.

31. The method of claim 25, further comprising applying heat to the mold and, before removing the bellows region from the mold, allowing the mold to cool.
